(19) United States
(12) Patent Application Publication
(10) Pub. No.: US 2026/0166256 A1
(43) Pub. Date: Jun. 18, 2026

(54) DEVICES, SYSTEM AND METHODS FOR MONITORING AND/OR CONTROLLING INFANT BREATHING

(71) Applicant: Prapela, Inc., Portland, ME (US)

(72) Inventors: David PAYDARFAR, Austin, TX (US); James NIEMI, Concord, MA (US); John KONSIN, S. Portland, ME (US); David MORRILL, Gorham, ME (US)

(21) Appl. No.: 19/345,924

(22) Filed: Sep. 30, 2025

Related U.S. Application Data
(60) Provisional application No. 63/733,253, filed on Dec. 12, 2024.

(51) Int. Cl. A61M 21/00

(52) U.S. Cl. CPC A61M 21/00; A61M 2021/0022; A61M 2021/0083

(57) ABSTRACT

Disclosed herein are devices, systems and methods for monitoring and/or controlling respiration in a subject, particularly an infant during sleep. Systems disclosed herein include a mattress and a wearable sensing device, capable of monitoring various physiological parameters of the subject, including blood oxygen level. The mattress provides various vibrational stimulation modes to the infant, including sub-arousal and/or arousal mode, depending on the monitored physiological parameters of the infant.

### CROSS-REFERENCES TO RELATED APPLICATIONS

This application is a U.S. Non-Provisional Application and claims priority to and benefit of U.S. Provisional Application Serial No. 63/733,253, filed on December 12, 2024, the content of which is incorporated herein in its entirety.

### TECHNICAL FIELD

This disclosure relates to devices, systems and methods for monitoring and improving infant respiration and/or respiratory related events during sleep. More particularly, the invention relates to a device, system and method which utilizes stochastic vibrational stimulation to regulate infant breathing and/or sleeping events.

### BACKGROUND

At risk infants are often monitored in a hospital for blood oxygen level, heart rate, movement, temperature, and/or respiration rate and alarms are set to alert the hospital staff when an infant stops breathing. While hospital procedures vary, a typical response to an alarm is for the hospital staff to nudge or rub the infant, which often results in regular breathing being re-stablished. If regular breathing is not re-established, ultimately the staff will wake the infant as a more aggressive response to re-establish regular breathing.

However, once infants leave the hospital and are in the home environment, the frequent monitoring and alarms are no longer available. This is even more problematic during infant sleep, where the parents or guardians are not always present at the infant’s bedside. Therefore, a system or device is needed not only for monitoring respiration rates of infants at home, but also for re-establishing respiration of the infant, without the necessity of parental intervention, while the infant sleeps.

### SUMMARY

Disclosed herein are devices, systems and methods for monitoring and/or controlling respiration in a subject, particularly an infant during sleep. Systems disclosed herein include a mattress and a wearable sensing device, capable of monitoring various physiological parameters of the subject, including blood oxygen level. The mattress or wearable provides stochastic vibrational stimulation to the infant in a sub-arousal or arousal mode, depending on the monitored physiological parameters of the infant.

Disclosed herein is a system for monitoring and/or controlling respiration in a subject is disclosed. The system includes:

a mattress or wearable configured to provide vibrational stimulation to the subject;

a device configured to sense and/or monitor physiological data of the subject; and

a processor configured to gather the physiological data from the device.

The processor takes the physiological data gathered from the device and uses it to determine vibrational modes of the mattress.

In one example, the wearable device is to sense and/or monitor physiological parameters and/or data selected from blood oxygen level, heart rate or pulse rate, respiration rate, temperature, movement, weight, subject location, sound, or a combination thereof. In one embodiment the wearable device includes a pulse oximeter sensor, a hear rate sensor, a temperature sensor, a respiratory sensor, a movement sensor, an accelerometer, a radar, or a device which includes a combination of these sensing capabilities.

Also disclosed, are methods for monitoring and/or controlling respiration of a subject. The method comprise the steps of:

monitoring physiological parameters of the subject;

determining if a critical event has occurred or is predicted, based on the monitored physiological parameters of the subject;

maintaining or activating a sub-arousal vibrational mode if no critical event has occurred or is predicted; and

maintaining or activating an arousal vibrational mode if a critical event has occurred or is predicted.

The step of monitoring physiological parameters is accomplished through use of the sensing device, disclosed and detailed in embodiments that follow.

### Selected definitions and nomenclature

As used herein, the term “stochastic” in relation to stimulation and/or vibration, refers to randomly determined and unpredictable stimulation and/or vibration, having a random probability distribution or pattern.

As used herein, the term “arousal mode” refers to a mode of provided vibrational stimulation, which is configured to arouse the subject from sleep.

As used herein, the term “sub-arousal mode” refers to a mode of provided vibrational stimulation, which is configured to keep the subject asleep, or to not arouse a subject from sleeping.

As used herein, the term “monotonous” in relation to vibrational stimulation refers to continuous and unchanging vibration.

Various terms are used to refer to particular system components. Different companies may refer to a component by different names – this document does not intend to distinguish between components that differ in name but not function. In the following discussion and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The term “about” is used in conjunction with numeric values to include normal variations in measurements as expected by persons skilled in the art, and is understood to have the same meaning as “approximately” and to cover a typical margin of error, such as ±15%, ±10%, ±5%, ±1%, ±0.5%, or even ±0.1% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise. As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5% or even 1%) detectable activity or amount.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” “inside,” “outside,” “contained within,” “superimposing upon,” and the like, may be used herein. These spatially relative terms can be used for ease of description to describe one element’s or feature’s relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

For a detailed description of example embodiments, reference will now be made to the accompanying drawings in which:

FIG. 1 generally illustrates a perspective view of a device in accordance with embodiments of the present disclosure;

FIG. 2 generally illustrates an exploded view of a device, in accordance with embodiments of the present disclosure;

FIG. 3 generally illustrates a block diagram of components of a system, in accordance with embodiments of the present disclosure;

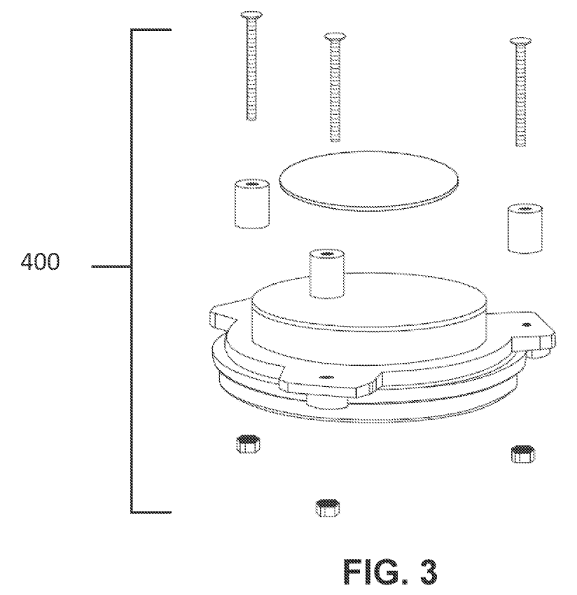

FIG. 4 generally illustrates a block diagram of another embodiment of a system, in accordance with the present disclosure;

FIG. 5 generally illustrates a perspective view of a device for monitoring and analysis of bodily emissions, in accordance with embodiments of the present disclosure;

FIG. 6 generally illustrates a method of monitoring and/or controlling respiration of a subject, in accordance with embodiments of the present disclosure.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Disclosed herein are devices, systems and methods for monitoring and/or controlling respiration of a subject, particularly while the subject is sleeping.

In one embodiment, a system for monitoring and/or controlling respiration in a subject is disclosed. The system includes:

a mattress or wearable configured to provide vibrational stimulation to the subject;

a device configured to sense and/or monitor physiological data of the subject; and

a processor configured to gather the physiological data from the device.

The processor takes the physiological data gathered from the device and uses it to determine vibrational modes of the mattress. The vibrational modes disclosed herein include at least an arousal mode and a sub-arousal mode, the particulars of which are described in more detail in sections below.

In certain embodiments, the device configured to sense and/or monitor physiological data and/or vibrate can be a wearable device which is attached to the subject. In other embodiments, the device can be incorporated as part of the mattress or configured to be attached to the mattress. In one example, the wearable device is configured to sense and/or monitor physiological parameters and/or data selected from blood oxygen level, heart rate or pulse rate, respiration rate, temperature, movement, weight, subject location, sound, or a combination thereof, and can vibrate or produce a tone. In certain embodiments the wearable device includes a pulse oximeter sensor, a hear rate sensor, a temperature sensor, a respiratory sensor, a movement sensor, a vibration device, a loudspeaker or a device which includes a combination of these sensing capabilities.

Examples of wearable monitoring devices and wearable pulse oximeters which are usable with the system of this invention include those disclosed in patent publications US US2017/0245791A1, titled “Wireless infant health monitor”, the relevant sections of which are incorporated herein by reference. The device is attachable to the subject of interest. In one example, the subject is an infant and the device is wearable on an appendage of the infant, such as the infant’s foot, hand, arm, leg or torso.

In one embodiment, the device monitors blood-oxygen level of the infant. Infant breathing disruptions, for example when an infant holds their breath for a brief period of time, can cause the infant’s blood oxygen concentration to drop. The drop in oxygen, when monitored for a specific threshold, can be used as an indicator that the infant’s respiration has ceased temporarily. At this time, a processor which communicates with the mattress and the wearable device, determines a vibration mode of the mattress or wearable device.

At least two vibrational modes are determined and actioned by the processors. One is a sub-arousal vibrational mode and another is an arousal vibrational mode. The sub-arousal mode is configured to keep the infant asleep, while the arousal vibrational mode is configured to provide a higher degree of vibration so as to wake the infant via movement and or tone generated by the mattress and trigger respiration, if the infant has experienced a brief cessation in breathing. The wearable device, the mattress, or a combination of them can provide the vibration.

In one embodiment, the vibrational stimulation provided to the infant in either arousal or sub-arousal mode is a stochastic vibrational stimulation, which means that the amplitude and frequency of the vibration stimulation is random and does not follow a particular repeating pattern. In another embodiment, the vibration stimulation provided is monotonous vibrational stimulation. The monotonous vibrational stimulation has a frequency in the range of 30 Hz to 15 kHz, with an amplitude ranging between 10 µm to 10 mm. In an alternate embodiment, a hybrid vibrational stimulation is provided, which comprises both monotonous and stochastic vibrational stimulation. The stochastic and monotonous vibrational stimulation can be provided simultaneously or in a sequential pattern, which can be randomized or predetermined sequence. In one example, monotonous vibrational stimulation is provided to aid an infant into sleep, and once asleep a switch is made to stochastic vibration to improve respiration.

In one embodiment, during a sub-arousal mode, the stochastic vibrational stimulation to the subject that is delivered to the subject has a vertical displacement of 10-20 µm ± 15% displacement root mean squared (DRMS). In this mode, 50% or greater of the vibration power spectral density is between 30-60 Hz, and 90% or greater of the vibration power spectral density is between 0-90Hz.

In another embodiment, during arousal mode, the stochastic vibrational stimulation that is delivered to the subject has a vertical displacement of 20 µm – 40 mm, and a vibrational frequency range between 90Hz to 9kHz. The amplitude and frequency are stochastic within this range. It should be noted that this frequency and amplitude range are audible and can be heard. In other embodiments, audible tones and/or melodies can be generated by the device as needed. For example, randomized signals with a different power spectrum than stochastic vibration can be utilized. As time passes and the infant ages, less stochastic vibration may be used and other types of vibrations and/or noise types may be required for an effective sleep aid or arousal aid.

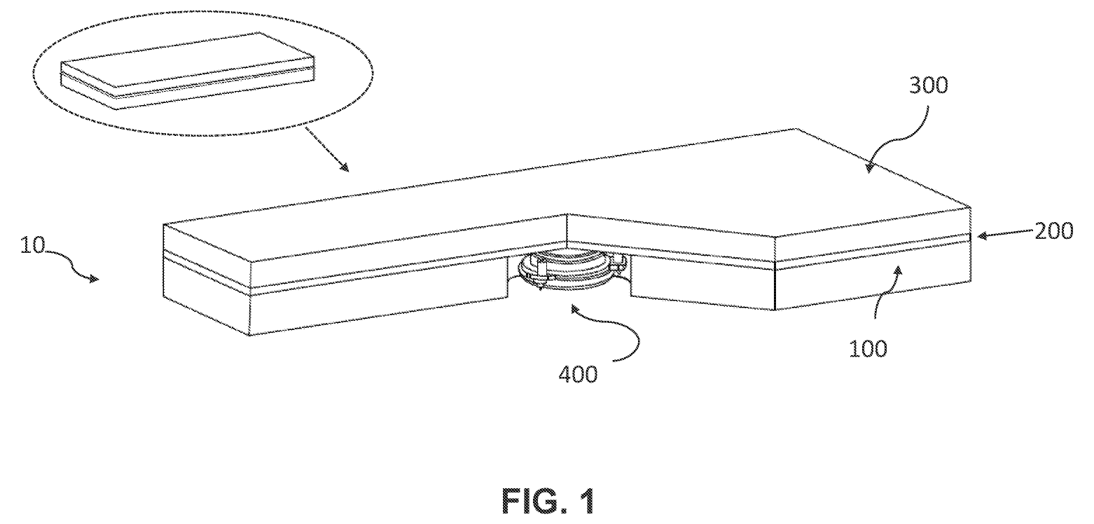

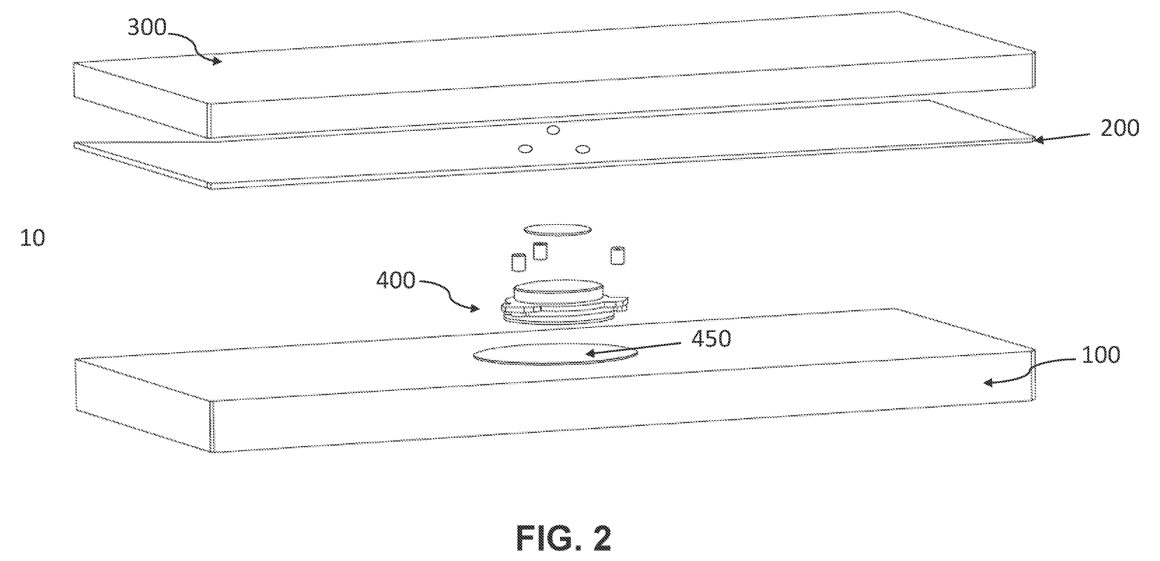

Shown in FIGS. 1 and 2 is an embodiment of a mattress, as disclosed in the present invention. The mattress 10 includes the following components:

a bottom substrate 100;

an intermediate vibration board 200;

a top substrate 300; and

at least one vibration generator 400.

The vibration generator 400, can be a transducer in one embodiment, and is located within a centrally located recess 450 of the bottom substrate 100. The transducer 400 attaches to the intermediate vibration board 200 through fasteners, such as screws, or any other known fastening means. The transducer is configured to provide vibration to the intermediate vibration board 200, which is in contact with the top substrate and thereby the infant resting on the top substrate of the mattress 10. More than one vibration generators may be used in various locations within the mattress.

The top and bottom substrates may be composed of a foam, rubber or fabric material. The intermediate vibration board can be composed of a polymeric material or other suitable materials. A surface covering may also be provided to encase the components of the mattress 10 to make it a comfortable sensory material for an infant to rest on. The dimensions and size of the mattress can be standardized to fit any size of crib, mattress, cot, bassinet, bedside sleeper, baby box, playard on a per use basis. Preferably the size of the mattress allows for less than a 1 inch clearance around each edge of the infant bed its placed in. Therefore, the mattress is designed to fit snugly within the infant bed.

In one example, the processor can be incorporated in a controller which is communicatively coupled to the mattress and/or wearable device. In one embodiment, the processor is configured to gather the physiological data from the wearable device attached to the subject. The gathered data from the device is then used by the processor to determine the vibrational mode. As noted earlier, the vibrational mode includes at least a sub-arousal mode or an arousal mode. As an example, if the processor gathers oxygen-level data from the device, and determines that the infant’s oxygen level has dropped below a predetermined threshold level, then the processor will actuate the transducer to enter arousal mode, so as to force the infant to waken through a higher degree of vibration and/or tone provided. Actuating arousal mode vibration will induce the infant potentially to waken or stir and restore a more normal respiration rate. Alternatively, if the processor determines that at any given time the monitored parameter (in this example, blood oxygen level) is within an acceptable predetermined threshold value, then the processor will determine to maintain or initiate a sub-arousal vibrational mode. The sub-arousal vibration mode is designed to provide stochastic vibrational stimulation which ensure the infant remains asleep. This mode of vibration (at the disclosed frequencies) is configured to provide stimulation to the infant, which aids in preventing cessation of respiration. Stochastic vibrational stimulation, in the sub-arousal mode is also understood to aid in improved sleep and duration of sleep of the infant.

The controller housing the processor may be wireless, or physically attached to the mattress. The controller may also include buttons that a parent or guardian may actuate to control the vibration modes and operation of the mattress (i.e. turning the vibration modes or monitoring on/off). It may contain a display or touchscreen display to control the modes and operation, and provide status reports. The vibrational modes can be turned on or off with the controller for a predetermined amount of time, or can be configured to remain on continuously, until a physiological parameter causes the processor to change the vibrational mode.

In further embodiments, the processor is also configured to collect data from the mattress and/or sensing device and transmit the data to an artificial intelligence database system. Based on the transmitted data, the artificial intelligence database system is configured to customize and/or optimize the stochastic vibrational stimulation provided to the subject. The transmitted data can be analyzed by the AI system to understand patterns of the infant, predict patterns, and potentially prevent critical events from occurring based on those patterns. Critical events include a cessation of respiration, or any measured physiological parameter being below or above a predetermined safe threshold. All of this can be reported via a display or smart phone.

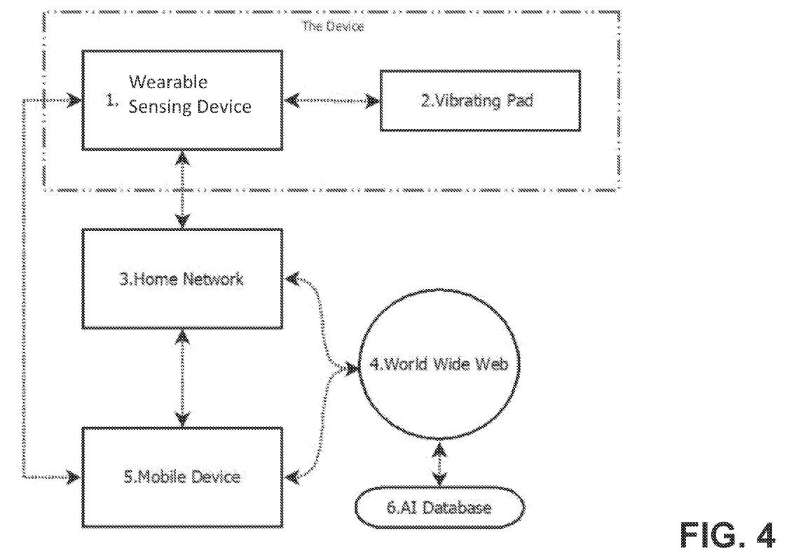

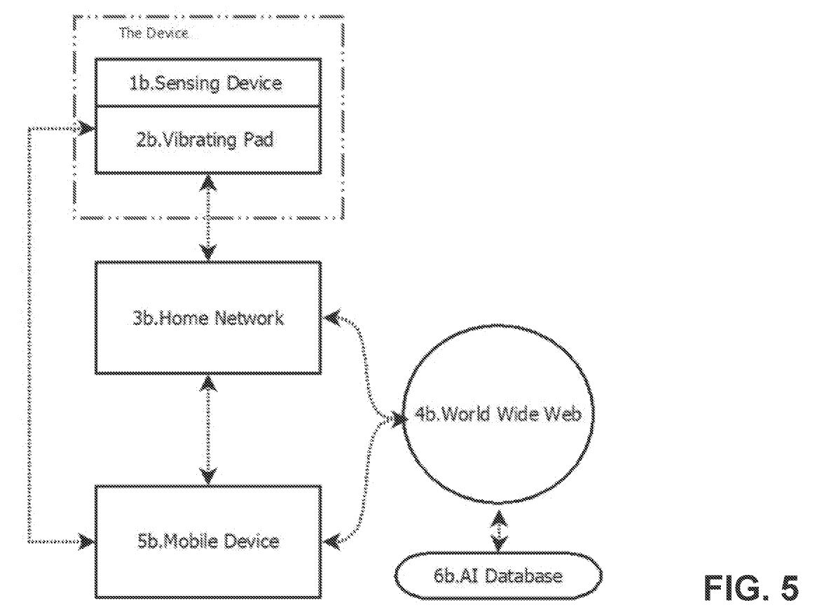

Shown in FIG. 4 and FIG. 5 are various embodiments detailing components of the infant monitoring systems disclosed herein. In the embodiment shown in FIG. 4 the mattress (labeled as “vibrating pad”) and the wearable sensing device are two separate components, which are configured to communicate with each other through a processor. Both the wearable device can wirelessly communicate with a home network and/or mobile device of a user. Both the home network and/or the mobile device can communicate with the AI database system disclosed herein.

In the embodiment shown in FIG. 5, the mattress and the sensing device are composed as components of a single device. For example, the sensing capabilities (i.e., various sensors) can be incorporated in the mattress itself, instead of being a separate wearable device attachable to the infant/subject.

In one embodiment, the vibration generator(s) and the vibration board can be molded together as a single piece. The vibration generator(s) may also integrated into an existing bassinet/crib platforms. The mattress pad may also have a charger or wireless base station integrated to the pad, or it can be coupled externally and paired wirelessly.

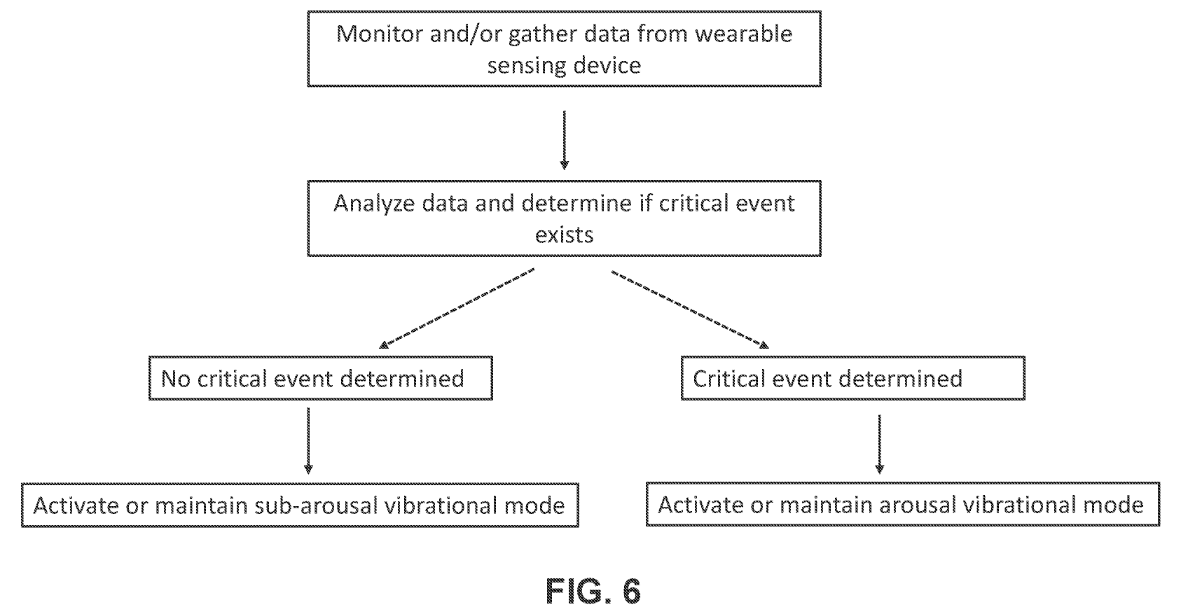

Also disclosed, are methods for monitoring and/or controlling respiration of a subject as shown in FIG. 6. The method comprise the steps of:

monitoring physiological parameters of the subject;

determining if a critical event has occurred or is predicted to occur, based on the monitored physiological parameters of the subject;

maintaining or activating a sub-arousal vibrational mode if no critical event has occurred or is predicted; and

maintaining or activating an arousal vibrational mode if a critical event has occurred or is predicted.

The step of monitoring physiological parameters is accomplished through use of the sensing device, disclosed and detailed in embodiments above. As already noted, the sensing device can comprise a pulse oximeter sensor, a hear rate sensor, a temperature sensor, a respiratory sensor, a movement sensor, a camera, radar, and/or a combination thereof.

In certain embodiments, maintaining or activating a sub-arousal vibration mode includes providing stochastic vibrational stimulation to the subject with via the mattress, wherein the stimulation includes a vertical displacement of 10-20 µm ± 15% displacement root mean squared (DRMS).

Maintaining or activating an arousal vibration mode includes providing stochastic vibrational stimulation to the subject with a vertical displacement of 20 µm – 40 mm, and a vibrational frequency in the range of 90Hz to 9kHz.

Monitoring physiological parameters of the subject includes sensing and/or monitoring physiological data selected from blood oxygen level, heart rate or pulse rate, respiration rate, temperature, movement, or a combination thereof. This data is analyzed by the processor to determine if a critical event has occurred. The critical event can include, for example, a cessation of respiration of the infant. This may be determined, in one instance, by monitoring a drop of oxygen level of the infant below a predetermined threshold. Similarly, other parameters may be monitored to identify a critical event, such as respiration rate, heart rate, movement etc. A remote device may also be alerted if a critical event has occurred. For example, a remote device may include a wireless device, such as a mobile phone. In this way, a parent or caretaker of the infant can be alerted in changes of physiological parameters being monitored. The user of the mobile device can have the capability to remotely activate the vibrational modes of the mattress.

The method also includes transmitting the collected physiological parameter data to an artificial intelligence database system. The artificial intelligence database system is configured to customize and/or optimize the stochastic vibrational stimulation provided to the subject.

With regards to the artificial intelligence (AI) processing capability, in some embodiments, the AI based analysis system may include one or more machine learning models that are trained to perform any of the techniques or methods disclosed herein. The one or more machine learning models may be generated by a training engine and may be implemented in computer instructions executable by one or more processing devices of the training engine and/or servers. To generate the one or more machine learning models, the training engine may train the one or more machine learning models.

The training engine may be a rackmount server, a router computer, a personal computer, a portable digital assistant, a smartphone, a laptop computer, a tablet computer, a netbook, a desktop computer, an Internet of Things (IoT) device, any other desired computing device, or any combination of the above. The training engine may be cloud-based or a real-time software platform, and it may include privacy software or protocols, and/or security software or protocols.

Using training data that includes training inputs and corresponding target outputs, the one or more machine learning models may refer to model artifacts created by the training engine. The training engine may find patterns in the training data wherein such patterns map the training input to the target output, and generate the machine learning models that capture these patterns. The one or more machine learning models may comprise, e.g., a single level of linear or non-linear operations (e.g., a support vector machine [SVM]) or the machine learning models may be a deep network, i.e., a machine learning model comprising multiple levels of non-linear operations. Examples of deep networks are neural networks including generative adversarial networks, multimodal large language models (LLMs), visual transformer models, convolutional neural networks, recurrent neural networks with one or more hidden layers, and fully connected neural networks (e.g., each neuron may transmit its output signal to the input of the remaining neurons, as well as to itself). For example, the machine learning model may include numerous layers and/or hidden layers that perform calculations (e.g., dot products) using various neurons.

In one embodiment, the one or more machine learning models may be trained by training input data which includes labeled blood oxygen level values or patterns, heart rates values or patterns, temperature values or patterns, a combination thereof, and so on.

In a further embodiment, input data can include labeled segments of a session video with one or more labels that describe the action that is occurring in the video during that interval of time (e.g., active urine voiding, active fecal excretion, flushing, toilet paper present, blood present, and so on).

The input labeled data can be mapped or correlated to specific parameters which are indicative of certain critical events occurring.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, privacy, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A system for monitoring and/or controlling respiration in a subject, the system comprising:
a mattress configured to provide vibrational stimulation to the subject;
a device configured to sense and/or monitor physiological data of the subject; and
a processor configured to gather the physiological data from the device;
wherein the physiological data gathered from the device is used to determine vibrational modes of the mattress.

2. The system of claim 1, wherein the vibrational modes of the mattress comprise at least a sub-arousal mode and an arousal mode.

3. The system of claim 2, wherein the sub-arousal mode and arousal mode comprise stochastic vibrational stimulation, monotonous vibrational stimulation, or hybrid vibrational stimulation.

4. The system of claim 2, wherein the sub-arousal mode is configured to provide stochastic vibrational stimulation to the subject with a vertical displacement of 10-20 µm ± 15% displacement root mean squared (DRMS).

5. The system of claim 2, wherein the sub-arousal mode is configured to provide stochastic vibrational stimulation to the subject with 50% or greater of the vibration power spectral density is between 30-60 Hz, and 90% or greater of the vibration power spectral density is between 0-90Hz.

6. The system of claim 2, wherein the arousal mode is configured to provide vibrational stimulation to the subject with a vertical displacement of 20 µm – 40 mm.

7. The system of claim 2, wherein the arousal mode is configured to provide vibrational stimulation to the subject at a vibrational frequency of 90Hz to 9kHz.

8. The system of claim 1, wherein the device is configured to sense and/or monitor physiological data selected from blood oxygen level, heart rate or pulse rate, respiration rate, temperature, movement, weight, subject location, sound, or a combination thereof.

9. The system of claim 1, wherein the device comprises a pulse oximeter sensor, a heart rate sensor, a temperature sensor, a respiratory sensor, a movement sensor, an accelerometer, a microphone, a camera, a weight sensor, location/position sensor, a radar, and/or a combination thereof.

10. The system of claim 1, wherein the mattress comprises:
a bottom substrate;
an intermediate vibration board;
a top substrate; and
at least one vibration generator.

11. The system of claim 10, wherein the at least one vibration generator is located in a recess within the bottom substrate.

12. The system of claim 1, wherein the processor is further configured collect data from the mattress and/or sensing device and transmit the data to an artificial intelligence database system.

13. The system of claim 1, wherein the physiological data gathered from the device is transmitted to an artificial intelligence database system, which then determines vibrational modes of the mattress provided to the subject.

14. A method of monitoring and/or controlling respiration of a subject, the method comprising:
monitoring physiological parameters of the subject;
determining if a critical event has occurred or is predicted to occur, based on the monitored physiological parameters of the subject;
activating or maintaining a sub-arousal vibrational mode if no critical event has occurred or is not predicted; and
activating or maintaining an arousal vibrational mode if a critical event has occurred or is predicted.

15. The method of claim 14, wherein the step of monitoring physiological parameters comprises monitoring physiological data from a device configured to sense and/or monitor physiological data of the subject.

16. The method of claim 15, wherein the device comprises a pulse oximeter sensor, a heart rate sensor, a temperature sensor, a respiratory sensor, a movement sensor, a microphone, a camera, a weight sensor, a location/position sensor, a radar, and/or a combination thereof.

17. The method of claim 14, wherein the step of activating or maintaining a sub-arousal mode comprises providing stochastic vibrational stimulation to the subject with a vertical displacement of 10-20 µm ± 15% displacement root mean squared (DRMS).

18. The method of claim 14, wherein the step of activating or maintaining a sub-arousal mode comprises providing comprises providing stochastic vibrational stimulation to the subject with 50% or greater of the vibration power spectral density is between 30-60 Hz, and 90% or greater of the vibration power spectral density is between 0-90Hz.

19. The method of claim 14, wherein the step of activating or maintaining an arousal mode comprises providing stochastic vibrational stimulation to the subject with a vertical displacement of 20 µm – 40 mm, at a vibrational frequency of 90Hz to 9kHz.

20. The method of claim 14, wherein the step of determining if a critical event has occurred or is predicted comprises analysis of physiological data gathered from the device by an artificial intelligence database system, which then determines vibrational modes of the mattress provided to the subject.
